(19) United States
(12) Patent Application Publication
(10) Pub. No.: US 2005/0113741 A1
(43) Pub. Date: May 26, 2005

(54) Device for controlling fluid flow in an aspiration system

(75) Inventors: David Huang, South Pasadena, CA (US); John D. Hall, Grand Rapids, MI (US); Mark S. Goodin, Solon, OH (US); David L. Thompson, Houston, TX (US)

Correspondence Address: TAROLLI, SUNDHEIM, COVELL & TUMMINO L.L.P., 526 SUPERIOR AVENUE, SUITE 1111, CLEVEVLAND, OH, 44114, US

(73) Assignee: The Cleveland Clinic Foundation

(21) Appl. No.: 10/965,314

(22) Filed: Oct. 14, 2004

Related U.S. Application Data
(60) Provisional application No. 60/511,442, filed on Oct. 15, 2003.
(60) Provisional application No. 60/513,493, filed on Oct. 22, 2003.

(51) Int. Cl. A61M 1/00

(52) U.S. Cl. 604/35

(57) ABSTRACT

A device for, when connected to a vacuum system, conducting aspiration fluid away from a surgical site includes a conduit having a portion adapted for insertion into the surgical site. The device also includes a flow limiter interposed between the vacuum system and the conduit and in fluid communication with the conduit. The flow limiter has at least two orifices arranged in series and a flow path interconnecting the at least two orifices. The flow path has a flow area that is greater than a flow area of each one of the at least two orifices. Aspiration fluid flows through the conduit and the flow limiter in response to a pressure drop between the surgical site and the vacuum system. The flow limiter produces a non-linear relationship between a rate of aspiration fluid flow and the pressure drop.

### RELATED APPLICATIONS

This application claims the benefit of U.S. Provisional Application Nos. 60/511,442, filed Oct. 15, 2003 and 60/513,493, filed Oct. 22, 2003 and is incorporated herein by reference.

### TECHNICAL FIELD

The present invention relates to a device for controlling fluid flow in an aspiration system. More particularly, the present invention relates to a device for controlling fluid flow in an aspiration system for use during phacoemulsification.

### BACKGROUND OF THE INVENTION

A cataract is an opacity of the lens of an eye that results in impaired vision or blindness. Phacoemulsification is a surgical procedure in which nucleus, epinuclear material, and cortical material, are removed from the lens. After phacoemulsification is performed, only the lens capsule of the lens remains in the eye. During phacoemulsification, an incision is made in the cornea of the eye. An end portion of a tool is inserted into the fluid-filled anterior chamber of the eye. The inserted end portion of the tool is used for emulsifying the portions of the lens. Typically, ultrasonic energy moves the end portion of the tool relative to the lens for emulsifying the portions of the lens.

An aspiration system is used during phacoemulsification. The aspiration system includes a vacuum system that provides a negative pressure. The end portion of the tool that is used for emulsifying the portions of the lens is tubular and includes a lumen through which the negative pressure acts. The negative pressure is used to hold the portions of the lens against the end portion of the tool and to remove emulsified fragments from the anterior chamber of the eye.

An irrigation system is also used during phacoemulsification. The irrigation system provides fluid into the anterior chamber of the eye for maintaining fluid volume and pressure in the anterior chamber. If the aspiration system overwhelms the irrigation system, i.e., removes more fluid than the irrigation system supplies, the fluid pressure in the anterior chamber of the eye may drop and the anterior chamber of the eye may collapse. A collapse of the anterior chamber of the eye may result in damage to eye structures, such as the cornea, iris, and lens capsule. It is desirable to control the flow in the aspiration system so as to prevent the aspiration system from overwhelming the irrigation system.

One method of controlling the flow in the aspiration system is to reduce the negative pressure of the aspiration system. However, reduced negative pressure results in a slower, more difficult procedure since the negative pressure is used not only to aspirate the lens fragments but also to hold the portions of the lens to the end portion of the tool and help to breakup the lens material. Therefore, it is desirable to control the flow in the aspiration system while maintaining negative pressure through the lumen in end portion of the tool.

### SUMMARY OF THE INVENTION

The present invention relates to a device for, when connected to a vacuum system, conducting aspiration fluid away from a surgical site. The device comprises a conduit including a portion adapted for insertion into the surgical site. The device also comprises a flow limiter interposed between the vacuum system and the conduit and in fluid communication with the conduit. The flow limiter has at least two orifices arranged in series and a flow path interconnecting the at least two orifices. The flow path has a flow area that is greater than a flow area of each one of the at least two orifices. Aspiration fluid flows through the conduit and the flow limiter in response to a pressure drop between the surgical site and the vacuum system. The flow limiter produces a non-linear relationship between a rate of aspiration fluid flow and the pressure drop.

The present invention further provides a phacoemulsification system for, when connected to a vacuum system, conducting aspiration fluid and emulsified fragments of ocular lens components away from an eye. The phacoemulsification system comprises a conduit including a needle portion adapted for insertion into the surgical site, and a flow limiter interposed between the vacuum system and the conduit and in fluid communication with the conduit. The flow limiter has at least two orifice plates arranged in series and a spacer plate extending between the at least two orifice plates. Each of the orifice plates has an orifice extending therethrough. The spacer plate defines a flow path having a flow area that is greater than a flow area of each one of the at least two orifices. Aspiration fluid flows through the conduit and the flow limiter in response to a pressure drop between the surgical site and the vacuum system. The flow limiter produces a non-linear relationship between a rate of aspiration fluid flow and the pressure drop.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates an eye and a phacoemulsification system, including the device of the present invention, for performing a surgical procedure on the eye;

FIG. 2 is a cross-sectional view of the device of FIG. 1;

FIG. 2A is a view similar to FIG. 2 illustrating an alternate embodiment;

FIG. 3 is a view taken along line 3-3 in FIG. 2 and illustrates a first portion of the device;

FIG. 3A is a view taken along line 3A-3A in FIG. 2A;

FIG. 4 illustrates a second portion of the device of FIG. 2;

FIG. 5 is a first graph illustrating fluid flow as a function of system pressure drop for an exemplary flow limiter; and

FIG. 6 is a second graph illustrating fluid flow as a function of pressure drop across an exemplary flow limiter.

### DETAILED DESCRIPTION OF THE INVENTION

FIG. 1 illustrates an eye 10. The eye 10 includes a cornea 12 that partially defines a fluid-filled anterior chamber 14 of the eye. A lens 16 and an iris 18 are located within the anterior chamber 14.

FIG. 1 also illustrates a phacoemulsification system 20 for removing the lens 16 of the eye 10. The phacoemulsification system 20 includes an irrigation system 22, an aspiration system 24, and a phacoemulsification tool 26.

The irrigation system 22 includes a fluid supply 28, an irrigation conduit 30, and an irrigation needle 32. The fluid supply 28 is preferably saline. The irrigation conduit 30 connects the fluid supply 28 to the irrigation needle 32. The irrigation needle 32 includes a central lumen through which irrigation fluid, e.g., saline, flows. Irrigation fluid exits the lumen of the irrigation needle 32 at an opening 34 located at an end of the needle.

The aspiration system 24 includes a vacuum system 36 and an aspiration conduit 38. The vacuum system 36 creates a negative pressure (relative to atmospheric pressure). The aspiration conduit 38 includes a central passage and is connected to the vacuum system 36 so that the negative pressure acts through the passage of the aspiration conduit to create suction.

The phacoemulsification tool 26 includes a handpiece 40 and a needle 42 that extends outwardly of the handpiece. The needle 42 includes a proximal end 44 and a distal end 46. The proximal end 44 of the needle 42 includes external screw threads, indicated schematically at 48, for enabling the needle 42 to be connected to the handpiece 40. The distal end 46 of the needle 42 includes an angled tip 50. A lumen extends through the needle 42 from the proximal end 44 to the distal end 46. An opening 52 to the lumen is located at the angled tip 50 of the needle 42.

A distal end 54 of the handpiece 40 includes a recess 56 having internal threads. The recess 56 is sized for receiving the proximal end 44 of the needle 42. A conduit 58 extends through the handpiece 40 of the phacoemulsification tool 26. The conduit 58 includes a passage that terminates at the recess 56 in the distal end 54 of the handpiece 40. When the proximal end 44 of the needle 42 is screwed into the recess 56 in the distal end 54 of the handpiece 40, the passage of the conduit 58 is in fluid communication with the lumen of the needle 42.

The conduit 58 of the phacoemulsification tool 26 is connected to the aspiration conduit 38. As a result, negative pressure is passed through the conduit 58 and through the lumen of the needle 42 so that suction is present at the opening 52 of the angled tip 50 of the needle 42 of the phacoemulsification tool 26.

An emulsifier 60 is located within the handpiece 40 of the phacoemulsification tool 26. In one embodiment, the emulsifier 60 includes ultrasonic transducers that are operatively connected to the needle 42. The ultrasonic transducers cause the needle 42 to vibrate along its longitudinal axis, in a manner similar to a jackhammer. Vibration of the needle 42 results in high frequency movement of the angled tip 50 of the needle. The high frequency movement of the angled tip 50 of the needle 42 acts to fracture or emulsify the lens 16. Alternatively, the emulsifier 60 may utilize a laser, rotational oscillator, or water jet.

In another exemplary embodiment of the invention, the emulsifier 60 is removed from the phacoemulsification tool 26 and the suction from the aspiration system 24 acts through the lumen of the needle 42 to fragment the portions of the lens 16. Using the suction for fragmenting portions of the lens 16 is especially useful with soft cataracts. In addition to fragmenting the portions of the lens 16, the suction from the aspiration system 24 removes the fragmented portions of the lens 16 from the anterior chamber 14 of the eye.

The phacoemulsification system 20 illustrated in FIG. 1 also includes a flow limiter 62. The flow limiter 62 is located in the phacoemulsification tool 26 and forms part of the aspiration system 24 of the phacoemulsification system 20. The flow limiter 62 is in fluid communication with the conduit 58 of the phacoemulsification tool 26. As an alternative to locating the flow limiter 62 in the phacoemulsification tool 26, the flow limiter 62 may be connected to the aspiration conduit 38 in a location downstream of the phacoemulsification tool 26.

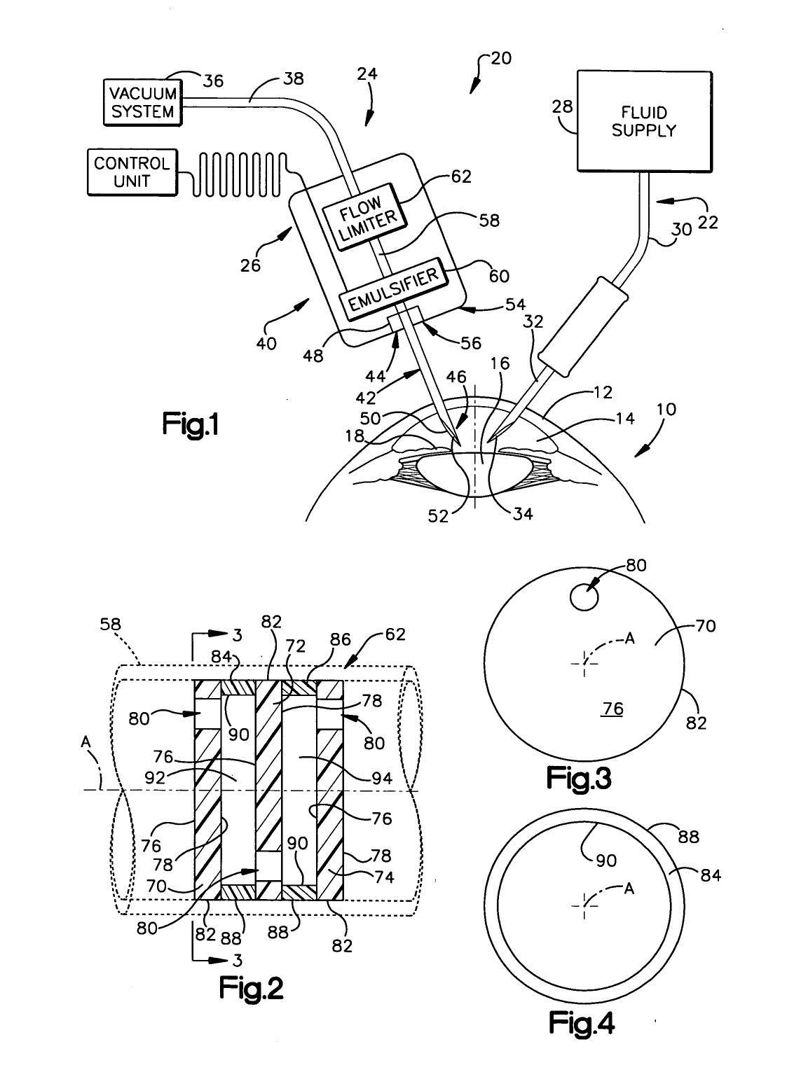

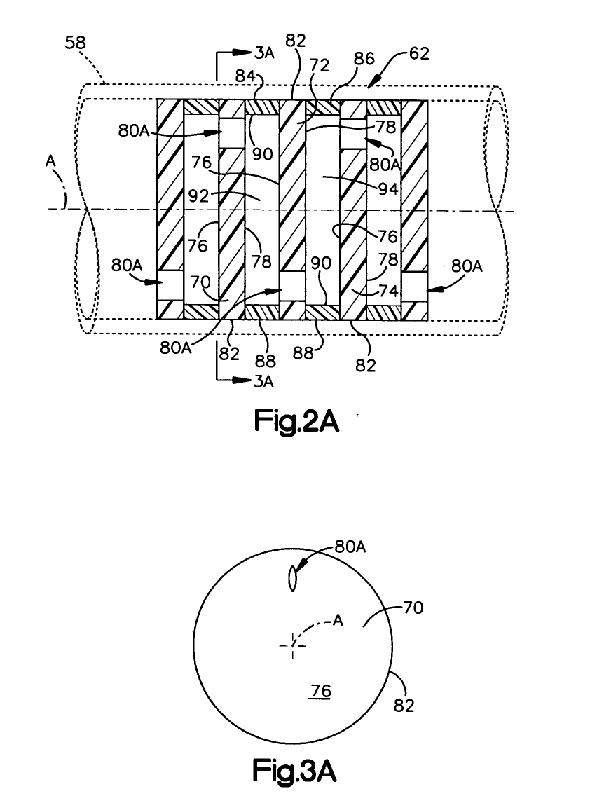

FIG. 2 illustrates a cut-away side view of the flow limiter 62. The flow limiter 62 is generally cylindrical and is centered on axis A. The flow limiter 62 may include two or more orifice plates. As illustrated in FIG. 2, the flow limiter 62 includes first, second, and third orifice plates 70, 72, and 74, respectively.

FIG. 3 illustrates a front view of the first orifice plate 70. The first, second, and third orifice plates 70, 72, and 74 are identical to one another. Each orifice plate 70, 72, and 74 is circular and includes oppositely disposed first and second side surfaces 76 and 78, respectively. A circular orifice 80 extends through each orifice plate 70, 72, and 74 from the first side surface 76 to the second side surface 78. The orifice plates 70, 72, and 74 and the orifice 80 of each orifice plate may be a shape other than circular, such as polygonal.

FIGS. 2A and 3A illustrate an alternate embodiment of the present invention in which the orifices are slits 80A. It is contemplated that using the slits 80A for orifices may decrease the chance of the orifices becoming plugged by emulsified fragments and may also make it easier to manufacture the orifice plates. It is further contemplated for the alternate embodiment of FIGS. 2A and 3A that the material of the orifice plates be a flexible enough to allow deformation of the slits so that emulsified fragments would pass through rather than become plugged in the slits 80A. Finally, as shown in FIG. 2A, it may be desirable to include additional stages of orifice plates and spacer plates when the orifices are slits.

The orifice 80 is located between axis A and an outer diameter 82 of the respective orifice plate 70, 72, and 74, and is thus radially offset from the centerline of the flow limiter 62. Alternatively, the orifices can be centered on axis A. Preferably, the diameter of each orifice 80 is substantially equal to the diameter of the lumen of the needle 42 of the phacoemulsification tool 26. As a result, each orifice 80 is large enough to resist occlusion by emulsified fragments that pass through the flow limiter 62.

The flow limiter 62 also includes first and second spacer plates 84 and 86, respectively. The number of spacer plates is one less than the number of orifice plates. FIG. 4 is a front view of the first spacer plate 84. Each of the first and second spacer plates 84 and 86 is annular and includes outer and inner diameters 88 and 90, respectively. The outer diameter 88 of each of the first and second spacer plates 84 and 86 is equal to an outer diameter 82 of the orifice plates 70, 72, and 74. The inner diameter 90 of the spacer plates 84 and 86 is located radially outwardly, relative to axis A, of any portion of the orifice 80 in the orifices plates 70, 72, and 74. The first and second spacer plates 84 and 86 have an axial length along axis A in FIG. 2 that is greater than a diameter of the orifices 80 so as to resist occlusion in the assembled flow limiter 62. The axial lengths of the spacer plates 84 and 86 may be changed to adjust a differential pressure of fluid flowing through the assembled flow limiter 62.

The first spacer plate 84 connects the first and second orifice plates 70 and 72 and spaces the second side surface 78 of the first orifice plate 70 from the first side surface 76 of the second orifice plate 72. Preferably, the second orifice plate 72 is rotated relative to the first orifice plate 70 so that, when connected by the first spacer plate 84, the orifice 80 in the first orifice plate 70 is located 180° relative to axis A from the orifice 80 in the second orifice plate 72. The inner diameter 90 of the first spacer plate 84, the second side surface 78 of the first orifice plate 70, and the first side surface 76 of the second orifice plate 72 collectively define a first cylindrical chamber 92.

The second spacer plate 86 connects the second and third orifice plates 72 and 74 and spaces the second side surface 78 of the second orifice plate 72 from the first side surface 76 of the third orifice plate 74. Preferably, the third orifice plate 74 is rotated relative to the second orifice plate 72 so that, when connected by the second spacer plate 86, the orifice 80 in the second orifice plate 72 is located 180° relative to axis A from the orifice 80 in the third orifice plate 74. Thus, the orifice 80 in the third orifice plate 74 is coaxial with the orifice 80 in the first orifice plate 70. The inner diameter 90 of the second spacer plate 86, the second side surface 78 of the second orifice plate 72, and the first side surface 76 of the third orifice plate 74 collectively define a second cylindrical chamber 94.

In one embodiment, the flow limiter 62 is made of a disposable plastic material. The flow limiter 62 may be packaged as a sterile, single use, disposable device. Prior to performing phacoemulsification, the sterile, single use, disposable flow limiter 62 is placed in the phacoemulsification tool 26 and the sterile, single use, disposable flow limiter 62 is discarded after phacoemulsification is complete. Alternatively, the flow limiter 62 may be a component of a disposable tubing set that includes the aspiration conduit 38.

With reference again to FIG. 1, the phacoemulsification system 20 of the present invention provides bi-manual irrigation/aspirations systems 22 and 24. During the phacoemulsification procedure, two small incisions are made to the cornea 12 of the eye 10. Each incision is approximately one to two millimeters in width. The irrigation needle 32 is inserted into the anterior chamber 14 of the eye 10 through one of the incisions and the needle 42 of the phacoemulsification tool 26 is inserted into the eye 10 through the other of the incisions. As a result, irrigation fluid passes into the anterior chamber 14 of the eye 10 through the first incision and fluid is aspirated from the eye 10 through the other incision.

Providing bi-manual irrigation/aspiration systems 22 and 24 enables improved surgical control. The location of the irrigation needle 32 and the needle 42 of the phacoemulsification tool 26 may be swapped or switched for providing easier access to all portions of the lens 16. Additionally, providing two small incisions, as opposed to one larger incision for both the irrigation/aspiration systems 22 and 24, reduces the risk of astigmatism.

During the phacoemulsification procedure, suction is provided through the lumen of the needle 42 of the phacoemulsification tool 26. The angled tip 50 of the needle 42 is maneuvered to a portion of the lens 16 and the suction holds the portion of the lens 16 to the angled tip 50. The portion of the lens 16 is then emulsified. For example, when the emulsifier includes ultrasonic transducers, vibration of the needle 42 moves the angled tip 50 relative to the eye 10 to emulsify the portion of the lens 16. The suction from the aspiration system 24 acting through the needle 42 of the phacoemulsification tool 26 removes the emulsified fragments from the anterior chamber 14 of the eye 10.

As stated above, a high negative pressure at the angled tip 50 of the needle 42 is desirable for holding the portion of the lens 16 to the angled tip 50 and for removing fragments from the anterior chamber 14 of the eye 10. However, care must be taken to ensure that the aspiration system 24 does not overwhelm the irrigation system 22.

A high level, i.e., a surge, of fluid flow through the aspiration system 24 is especially likely when an occlusion is released. For example, an emulsified fragment may temporarily occlude the lumen of the needle 42 of the phacoemulsification tool 26. When the needle 42 is occluded, the flow into the aspiration system 24 is stopped and the differential pressure across the occlusion increases. When the occlusion is released or breaks free, a flow surge into the aspiration system 24 may occur. The flow surge may result in the aspiration system 24 overwhelming the irrigation system 22 and the anterior chamber 14 of the eye 10 collapsing. The flow limiter 62 of the present invention helps to reduce flow surges while maintaining the ability to utilize high vacuum levels in the aspiration system 24.

Fluid flow through the conduit 58 of the phacoemulsification tool 26 is directed into the orifice 80 of the first orifice plate 70 of the flow limiter 62. Preferably, the flow limiter 62 provides a tortuous flow path having varying cross-sectional flow areas for generating flow turbulence in the fluid flow. The tortuous flow path through the flow limiter 62 results from the location of the orifices 80 of the orifice plates 70, 72, and 74. Fluid flow into the orifice 80 of the first orifice plate 70 is directed against the first side surface 76 of the second orifice plate 72 and into the first cylindrical chamber 92 of the flow limiter 62. The first side surface 76 of the second orifice plate 72 causes the fluid flow to change directions and the first cylindrical chamber 92 provides an increased flow area as compared to the orifice 80 of the first orifice plate 70. As a result, flow turbulence is created when the fluid flows through the orifice 80 of the first orifice plate 70 and into the cylindrical chamber 92.

Fluid flow from the first cylindrical chamber 92 then passes through the orifice 80 in the second orifice plate 72. Fluid flow through the second orifice plate 72 is directed against the first side surface 76 of the third orifice plate 74 and into the second cylindrical chamber 94. The first side surface 76 of the third orifice plate 74 causes the fluid flow to change directions and the second cylindrical chamber 94 provides an increased flow area as compared to the orifice 80 of the second orifice plate 72. As a result, turbulence is created when the fluid flows through the orifice 80 of the second orifice plate 72 and into the cylindrical chamber 94. Fluid flow from the second cylindrical chamber 94 passes through the orifice 80 in the third orifice plate 74 and eventually, into the conduit 38 of the aspiration system 24.

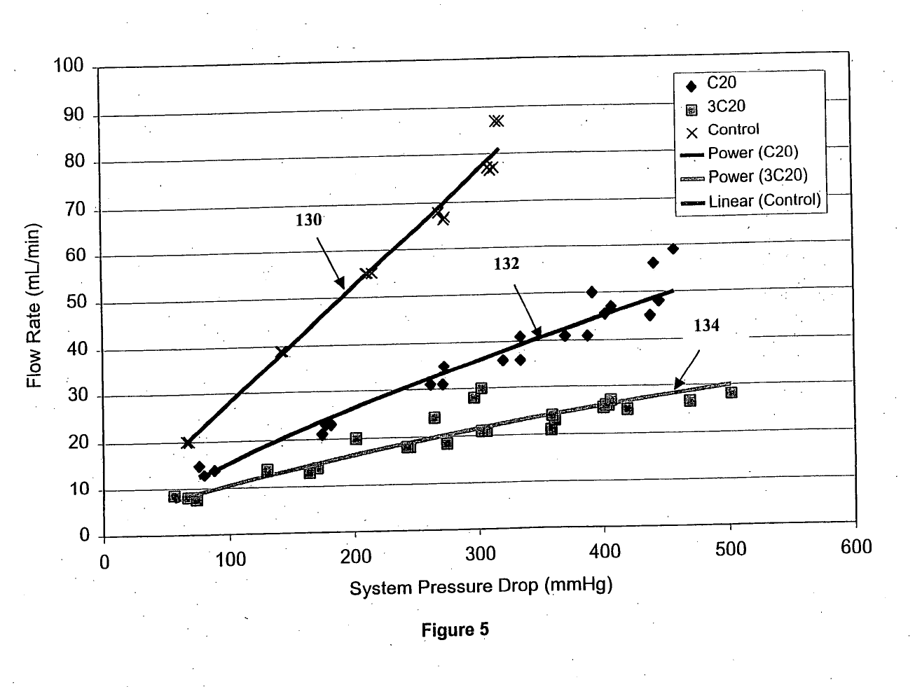

By generating turbulence in the fluid flow, the flow limiter 62 varies fluid flow in the aspiration system 24 non-linearly relative to the pressure drop between the surgical site, for example, the anterior chamber 14, and the vacuum system 36. FIG. 5 is a graph illustrating flow rate versus system pressure drop for 3/16 inch inner diameter control tubing, indicated at 130, for a single orifice plate having a 0.020 inch diameter orifice, indicated at 132, and for a flow limiter 62 having three orifice plates with serial 0.020 inch orifices, indicated at 134. As FIG. 5 illustrates, the flow limiter 62 provides high resistance to fluid flow at high system pressure drops and little resistance to fluid flow at lower system pressure drops. Thus, when subjected to a flow surge due to, for example, the release of an occlusion, the fluid flow rate through the flow limiter 62 is significantly less than the flow rate through a conduit having no flow limiter. As a result, the flow limiter 62 reduces the risk of the aspiration system 24 overwhelming the irrigation system 22 and causing a collapse of the anterior chamber 14 of the eye 10.

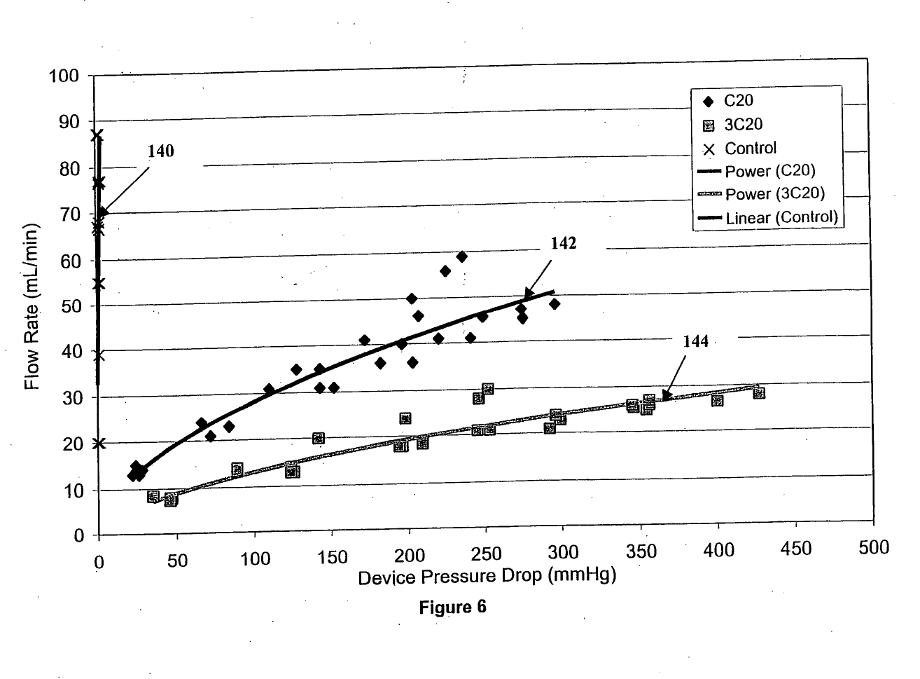

FIG. 6 is a graph illustrating fluid flow as a function of pressure drop across an exemplary flow limiter 62. This graph illustrates the non-linear flow versus pressure response of the flow limiter 62. When the flow limiter provides the primary source of resistance through the aspiration system 24 of the phacoemulsification 20, the non-linear relationship of system pressure drop to flow rate will emulate that illustrated in FIG. 6. Thus, the resistance of other portions of the aspiration system 24, such as the aspiration conduit 38, should be minimized to optimize the non-linear system response due to the flow limiter. In FIG. 6, flow rate versus system pressure drop for 3/16 inch inner diameter control tubing is indicated at 140, flow rate versus system pressure drop for a single orifice plate having a 0.020 inch diameter orifice is indicated at 142, and flow rate versus system pressure drop for a flow limiter 62 having three orifice plates with serial 0.020 inch orifices is indicated at 144.

As FIGS. 5 and 6 illustrate, the most effect way to adjust the non- linear flow resistance of the flow limiter 62 is to vary the number of orifice plates combined in series. By connecting several orifice plates in series, the flow limiter 62 can produce large non-linear flow resistance while maintaining relatively large orifices in the orifice plates that are not likely to become clogged by tissue fragments.

From the above description of the invention, those skilled in the art will perceive improvements, changes and modifications. For example, the orifices 80 of the flow limiter 62 may have varying diameters and shapes (e.g. elongated slots) for increasing the non-linear pressure versus flow relationship through the orifices. Each orifice plate 70, 72, and 74 may also include multiple orifices 80. Another similar configuration has orifice plates at 90° relative to each other. A series of divider plates, each with at least one orifice hole, span between the orifice plates described previously. The holes in each orifice and divider plate direct the flow through a maze-like pattern. Such improvements, changes and modifications within the skill of the art, are intended to be covered by the appended claims.

1. A device for, when connected to a vacuum system, conducting aspiration fluid away from a surgical site, the device comprising:
a conduit including a portion adapted for insertion into the surgical site; and
a flow limiter interposed between the vacuum system and the conduit and in fluid communication with the conduit, the flow limiter having at least two orifices arranged in series and a flow path interconnecting the at least two orifices, the flow path having a flow area that is greater than a flow area of each one of the at least two orifices;
wherein aspiration fluid flows through the conduit and the flow limiter in response to a pressure drop between the surgical site and the vacuum system, the flow limiter producing a non-linear relationship between a rate of aspiration fluid flow and the pressure drop.

2. The device of claim 1 wherein each of the at least two orifices has a diameter that is substantially equal to the diameter of the portion of the conduit that is adapted for insertion into the surgical site.

3. The device of claim 2 wherein the flow path connecting the at least two orifices comprises a spacer plate.

4. The device of claim 3 wherein the spacer plate has an axial length that is greater than the diameter of the at least two orifices.

5. The device of claim 1 wherein each of the at least orifices is located in and extends through an orifice plate.

6. The device of claim 5 wherein the flow limiter comprises at least two orifices plates that are separated by a spacer plate that defines the flow path.

7. The device of claim 6 wherein each of the at least two orifices is radially offset from a centerline of the flow limiter.

8. The device of claim 7 wherein the orifice in one of the at least two orifice plates is offset 180° relative to the centerline from the orifice in an adjacent one of the at least two orifice plates.

9. The device of claim 1 wherein the at least two orifices have a circular shape.

10. The device of claim 1 wherein each of the at least two orifices comprises a slit.

11. The device of claim 10 wherein each of the at least two slits is located in and extends through an orifice plate.

12. The device of claim 11 wherein the orifice plates are made from a flexible plastic material that is deformable to allow fragments to pass through the orifices.

13. The device of claim 12 wherein the flow limiter comprises at least two orifices plates that are separated by a spacer plate that defines the flow path.

14. The device of claim 13 wherein the flow limiter comprises at least three orifice plates separated by respective spacer plates.

15. A phacoemulsification system for, when connected to a vacuum system, conducting aspiration fluid and emulsified fragments of ocular lens components away from an eye, the phacoemulsification system comprising:
a conduit including a needle portion adapted for insertion into the surgical site; and
a flow limiter interposed between the vacuum system and the conduit and in fluid communication with the conduit, the flow limiter having at least two orifice plates arranged in series and a spacer plate extending between the at least two orifice plates, each of the orifice plates having an orifice extending therethrough, the spacer plate defining a flow path having a flow area that is greater than a flow area of each one of the at least two orifices;
wherein aspiration fluid flows through the conduit and the flow limiter in response to a pressure drop between the surgical site and the vacuum system, the flow limiter producing a non-linear relationship between a rate of aspiration fluid flow and the pressure drop.

16. The device of claim 15 wherein each of the at least two orifices has a diameter that is substantially equal to the diameter of the needle portion of the conduit.

17. The device of claim 16 wherein the spacer plate has an axial length that is greater than the diameter of the at least two orifices.

18. The device of claim 17 wherein each of the at least two orifices is radially offset from a centerline of the flow limiter.

19. The device of claim 18 wherein the orifice in one of the at least two orifice plates is offset 180° relative to the centerline from the orifice in an adjacent one of the at least two orifice plates.

20. The device of claim 15 wherein the at least two orifices have a circular shape.

21. The device of claim 15 wherein each of the at least two orifices comprises a slit.

22. The device of claim 21 wherein each of the at least two slits is located in and extends through a respective one of the orifice plates.

23. The device of claim 22 wherein the orifice plates are made from a flexible plastic material that is deformable to allow fragments to pass through the orifices.
